(19) United States
(12) Patent Application Publication
(10) Pub. No.: US 2024/0228953 A1
(43) Pub. Date: Jul. 11, 2024

(54) METHOD FOR PROMOTING PROLIFERATION AND PLURIPOTENCY FACTOR EXPRESSION OF STEM CELLS AND COMPOSITION PRODUCED USING THE SAME

(71) Applicant: BUDDHIST TZU CHI MEDICAL FOUNDATION, Hualien (TW)

(72) Inventors: WU LI Wei, Hualien (TW); Shinn-Zong LIN, Hualien (TW); Tzyy-Wen CHIOU, Hualien (TW); Horng-Jyh HARN, Hualien (TW); Peng-Yeong WOON, Hualien (TW)

(21) Appl. No.: 18/359,053

(22) Filed: Jul. 26, 2023

(30) Foreign Application Priority Data
Jan. 11, 2023 (TW) 112101219

(51) Int. Cl. C12N 5/079; C12N 5/00

(52) U.S. Cl. CPC C12N 5/0618; C12N 5/0018; C12N 2500/38; C12N 2501/15; C12N 2501/2306; C12N 2501/2308; C12N 2501/998; C12N 2501/999

(57) ABSTRACT

A method for promoting proliferation and enhancing expression of pluripotency factors of stem cells and a method for promoting proliferation of cells differentiated from the stem cells are provided in the present disclosure. The methods comprise culturing the stem cells in a medium comprising thrombospondin 1 (TSP1) and left-right determination factor 2 (LEFTY2). The present disclosure also provides a composition comprising a derivative of the stem cells produced by the methods of the present disclosure and a pharmaceutically or cosmetically acceptable carrier.

### CROSS-REFERENCE TO RELATED APPLICATIONS

This Application claims priority to Taiwan Patent Application No. 112101219, filed on Jan. 11, 2023. The entirety of the above-mentioned patent application is hereby incorporated by reference herein and made a part of this specification.

### BACKGROUND

### 1. Field of the Invention

The present disclosure relates to a use of the medium additive to promote proliferation and expression of pluripotency factors of stem cells, in particular, to promote proliferation of mesenchymal stem cells and the expression of their pluripotency factors. The present disclosure also relates to the use of the medium additive to promote proliferation of differentiated stem cells, in particular, to promote proliferation of nerve cells differentiated from such stem cells.

### 2. Description of the Prior Art(s)

Stem cells are cell types with the potential ability to differentiate and regenerate into complete individuals or various organs and tissues, and they exist in a variety of cells or tissues. Stem cells not only help to develop and differentiate into tissues and organs of an individual at the stage of embryonic development but also help to maintain the repair and renewal of tissues in mature individuals.

Stem cells can be classified into totipotent stem cells, pluripotent stem cells, multipotent stem cells, and unipotent stem cells based on their potential. Among them, pluripotent stem cells have the ability to develop into complete individuals, while multipotent stem cells are unable to form complete individuals compared to pluripotent stem cells; however, they still have the ability to develop into multiple tissues or organs. Multipotent stem cells are pluripotent stem cells that are down-specialized into stem cells that develop into specific tissues (e.g., hematopoietic stem cells) and have a limited ability to replicate. Unipotent stem cells can only form a single type of cell, but still have the ability to self-renew.

Stem cells play an important role in cell therapy and regenerative medicine because of their properties. However, in the current process of culturing stem cells, it is easy to lose their differentiation potential due to the rapid growth of cells towards differentiation. In addition, the cells differentiated from stem cells are not easy to maintain their growth in subsequent cultures. Therefore, it is still an urgent problem in the field to enhance the growth of stem cells while maintaining their differentiation potential, and to improve the cell death of differentiated stem cells as well.

### SUMMARY

In order to overcome the shortcomings mentioned above, the present disclosure provides a method for promoting proliferation of stem cells and maintaining the expression of pluripotency factors in the stem cells, comprising culturing the stem cells in a cell culture medium containing thrombospondin 1 (TSP1) and left-right determination factor 2 (LEFTY2).

In at least one embodiment of the present disclosure, the concentration of TSP1 is 10 ng/mL to 250 ng/mL, 20 ng/mL to 200 ng/mL, or 30 ng/mL to 150 ng/mL, such as but not limited to about 10 ng/mL, 20 ng/mL, 30 ng/mL, 40 ng/mL, 50 ng/mL, 60 ng/mL, 70 ng/mL, 80 ng/mL, 90 ng/mL, 100 ng/mL, 110 ng/mL, 120 ng/mL, 130 ng/mL, 140 ng/mL, ng/mL, 150 ng/mL, 160 ng/mL, 170 ng/mL, 180 ng/mL, 190 ng/mL, 200 ng/mL, 210 ng/mL, 220 ng/mL, 230 ng/mL, 240 ng/mL, 250 ng/mL.

In at least one embodiment of the present disclosure, the concentration of LEFTY2 is from 5 ng/mL to 120 ng/mL, 10 ng/mL to 100 ng/mL, or 10 ng/mL to 50 ng/mL, such as but not limited to about 5 ng/mL, 6 ng/mL, 7 ng/mL, 8 ng/mL, 9 ng/mL, 10 ng/mL, 20 ng/mL, 30 ng/mL, ng/mL, 40 ng/mL, 50 ng/mL, 60 ng/mL, 70 ng/mL, 80 ng/mL, 90 ng/mL, or 100 ng/mL.

In at least one embodiment of the present disclosure, the cell culture medium contains acetylcysteine and vitamin C. In some embodiments of the present disclosure, the concentration of acetylcysteine is 0.01 M to 1.0 M, 0.05 M to 0.8 M, or 0.1 M to 0.5 M, such as but not limited to about 0.01 M, 0.05 M, 0.1 M, 0.2 M, 0.3 M, 0.4 M, 0.5 M, 0.6 M, 0.7 M, 0.8 M, 0.9 M, 1.0 M; the concentration of vitamin C is 0.01 mM to 1.0 mM, 0.05 mM to 0.8 mM or 0.1 mM to 0.5 mM, such as but not limited to about 0.01 mM, 0.05 mM, 0.1 mM, 0.2 mM, 0.3 mM, 0.4 mM, 0.5 mM, 0.6 mM, 0.7 mM, 0.8 mM, 0.9 mM, 1.0 mM.

In at least one embodiment of the present disclosure, the stem cells are selected from the group consisting of induced pluripotent stem cells (iPSCs), mesenchymal stem cells (MSCs), adipose tissue-derived stem cells (ADSCs), bone marrow-derived stem cells (BMSCs), peripheral blood-derived stem cells (PBSCs), umbilical cord-derived stem cells (UCSCs), and amniotic fluid-derived stem cells (AFDSCs), but are not limited to the present disclosure.

In at least one embodiment of the present disclosure, the pluripotency factor is a transcription factor involved in stem cell pluripotency, such as but not limited to, a Yamanaka factor (e.g., Oct4, Sox2, Klf4, and/or c-Myc) or other factors (e.g., Lin28 and/or Nanog), etc. In some embodiments, the pluripotency factor is selected at least one from a group consisting of Oct4, Sox2, Klf4, c-Myc, Lin28, and Nanog, but is not limited to the present disclosure.

In at least one embodiment of the present disclosure, the stem cells have at least a 150% increase in growth (proliferation) compared to the control group, for example, about 150 to 160%, about 160 to 170%, about 170 to 180%, about 180 to 190%, about 190 to 200% or higher growth, wherein the control group is stem cells cultured in a medium that does not contain the medium additive. In at least one embodiment of the present disclosure, the stem cells have at least a 200% increase in pluripotent factors compared to the control group, for example., about 200 to 210%, about 210 to 220%, about 220 to 230%, about 230 to 240%, about 240 to 250% or greater increase in pluripotency, wherein the control group is a stem cell cultured in a medium that does not contain the medium additive. The comparison of the growth and pluripotency performance of the above stem cells and the control group is based on the same incubation time and equal initial cell numbers.

The present disclosure also provides a method for promoting proliferation of cells differentiated from stem cells, comprising culturing the stem cells or the cells differentiated from the stem cells in a cell culture medium comprising thrombospondin 1 (TSP1) and left-right determination factor 2 (LEFTY2).

In at least one embodiment of the present disclosure, the cells differentiated from the stem cells are nerve cells. In some embodiments, the medium additive reduces an expression of the inflammation-related factor or increases an expression of the anti-inflammation-related factor in the nerve cell. In some embodiments, the inflammation-related factor is selected from the group consisting of at least one of interleukin 6 (IL-6), interleukin 8 (IL-8), or macrophage migration inhibitory factor (MIF). At least one of the groups of IL-6, interleukin 8 (IL-8), or macrophage migration inhibitory factor (MIF), but is not limited to the present disclosure.

The present disclosure also provides a composition comprising a derivative of a stem cell and a pharmaceutically or cosmetically acceptable carrier, wherein the derivative is produced by a method comprising: providing a cell culture medium comprising thrombospondin 1 (TSP1) and left-right determination factor 2 (LEFTY2); culturing the stem cell in the cell culture medium; and obtaining the derivative from the cell culture medium.

In at least one embodiment of the present disclosure, the derivative is selected at least one from the group consisting of secretory protein bodies, conditioned medium, exosomes and extracellular vesicles or the combination thereof, and the stem cells are selected at least one from the group consisting of induced pluripotent stem cells, mesenchymal stem cells, fat stem cells, bone marrow stem cells, peripheral blood stem cells, umbilical cord stem cells and amniotic fluid stem cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows the results of cell growth of adipose-derived stem cells (ADSCs) cultured in media comprising different media additives in at least one embodiment of the present disclosure.

FIG. 1B shows the cell growth results of at least one embodiment of the present disclosure in which ADSCs were cultured in a medium comprising thrombospondin 1 (TSP1) and left-right determination factor 2 (LEFTY2) after 4 days.

FIG. 2A shows the variation of the expression of pluripotency factor Nanog in which ADSCs were cultured in a medium comprising different medium additives in at least one embodiment of the present disclosure, and the data are presented in a way that each group is standardized according to the performance of the control group.

FIG. 2B shows the variation of the expression of pluripotency factor OCT-4 in which ADSCs were cultured in a medium comprising different medium additives in at least one embodiment of the present disclosure, and the data are presented in a way that each group is standardized according to the performance of the control group.

FIG. 3 shows the influence on the expression of cell surface antigens CD73, CD90 and CD105 of culturing ADSCs in a medium comprising TSP1 and LEFTY2 in at least one embodiment of the present disclosure.

FIGS. 4A to 4C show cell type images of ADSCs differentiated into specific cells after being cultured in a medium comprising TSP1 and LEFTY2 in at least one embodiment of the present disclosure. FIG. 4A shows the cell type of adipocytes differentiated from ADSCs; FIG. 4B shows the cell type of the osteocytes differentiated from ADSCs; And FIG. 4C shows the cell type of chondrocytes differentiated from ADSCs.

FIG. 5 shows the influence on the production of exosomes of culturing ADSCs in a medium containing different medium additives and concentrations in at least one embodiment of the present disclosure.

FIG. 6A shows the influence on the cell survival of culturing nerve cells differentiated from induced pluripotent stem cells in a medium comprising different medium additives and concentrations in at least one embodiment of the present disclosure. FIG. 6B shows the image of the type of nerve cells cultured in the control group; FIG. 6C shows the image of the type of nerve cells cultured in the TSP1+LEFTY2 group.

FIG. 7 shows the influence on the expression of inflammatory factors of culturing damaged nerve cells in a medium containing different medium additives in at least one embodiment of the present disclosure.

### DETAILED DESCRIPTION

The following examples are used for illustrating the present disclosure. A person skilled in the art can easily conceive the other advantages and effects of the present disclosure, based on the disclosure of the specification. The present disclosure can also be implemented or applied as described in different examples. It is possible to modify or alter the following examples for carrying out this disclosure without contravening its scope, for different aspects and applications.

As used herein, the singular forms “a,” “an,” and “the” include plural referents, unless expressly and unequivocally limited to one referent. The term “or” is used interchangeably with the term “and/or,” unless the context clearly indicates otherwise. The terms “comprising”, “including”, “having” and the like mentioned herein are interpreted in an inclusive sense, that is, the meaning of “including but not limited to”.

The numeral ranges used herein are inclusive and combinable, any numeral value that falls within the numeral scope herein could be taken as a maximum or minimum value to derive the sub-ranges therefrom; for example, the range of values “0.01 M to 1.0 M” should be understood to include any subrange between the minimum value of 0.01 M and the maximum value of 1.0 M, for example, 0.01 M to 0.8 M, 0.05 M to 1.0 M, and 0.15 M to 0.45 M. In addition, a plurality of numeral values used herein can be optionally selected as maximum and minimum values to derive numerical ranges; for example, 0.05 M, 0.1 M, and 1.0 M can derive a range of values from 0.05 M to 0.1 M, 0.05 M to 1.0 M, and 0.1 M to 1.0 M.

As used herein, the term “about” generally referring to the numerical value meant to encompass variations of ±20%, ±10%, ±5%, ±1%, ±0.5%, or ±0.1% from a given value or range. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations. Alternatively, the term “about” means within an acceptable standard error of the mean when considered by one of ordinary skill in the art. Unless otherwise expressly specified, all of the numerical ranges, amounts, values and percentages such as those for quantities of materials, durations of time periods, temperatures, operating conditions, ratios of amounts, and the likes disclosed herein should be understood as modified in all instances by the term “about.”

As used herein, “stem cell” refers to a cell that has not yet differentiated and has the potential to regenerate into various tissues. The stem cells used in the specification are cells taken from individuals and tested in vitro, that is, cells in vitro or ex vivo. In some embodiments of the present disclosure, the individual is an animal, such as a mammal. In some embodiments of the present disclosure, the individual is human.

The term “pluripotency” as used herein refers to the property of differentiating cells to have various functions and types. Compared with “totipotency”, pluripotency does not allow cells to differentiate into placenta, but only has the ability to differentiate cells into various cells of the three germ layers outside the placenta, that is, cells with pluripotency cannot form complete individual. The term “pluripotency factor” refers to a substance involved in producing or maintaining pluripotency, wherein the substance can be a nucleic acid, a nucleic acid fragment, a gene, a protein or other molecules that can express the pluripotency biologically.

As used herein, the term “differentiation” refers to the change of stem cells tending to form cells with specific functions or shapes, including the process of stem cells transforming from growth-oriented to specialized. The differentiation process involves a series of successive changes in the cellular type, membrane potential, metabolic activity, and response to specific signals of the stem cells so that the differentiated stem cells acquire the corresponding type and specific cellular functions depending on the cellular spectrum of the tissue or organ in which they end up.

The stem cells described herein may be derived from individual organs or tissues including, but not limited to, bone marrow, peripheral blood, umbilical cord, placenta, and adipose. The stem cells described herein can be differentiated into individual adult cells such as adipocytes, osteoblasts, neurons, chondrocytes, muscle cells, cardiomyocytes, and pancreatic cells.

### EXAMPLES

### Example 1: TSP1 and LEFTY2 Medium Additive to Promote the Growth of Mesenchymal Stem Cells

In the embodiment of the present disclosure, Keratinocyte serum-free medium (K-SFM) which contains human recombinant epidermal growth factor (rEGF) and bovine pituitary extract (BPE) is taken as an example.

The K-SFM mentioned above was used as the control group, and the culture medium of following control group and test group were configure d as follows:
Control group: K-SFM;
Control group-2: K-SFM, 0.4M acetylcysteine and 0.2 mM vitamin C;
Control group-3: K-SFM, 0.4M acetylcysteine, 0.2 mM vitamin C and 10 ng/mL growth factor (fibroblast growth factor);
TSP1 (test group): control group-2 added 200 ng/mL TSP1;
LEFTY2 (test group): control group-2 added 50 ng/mL LEFTY2;
TSP1+LEFTY2 (test group): Control group-2 was supplemented with 200 ng/mL TSP1 and 50 ng/mL LEFTY2.

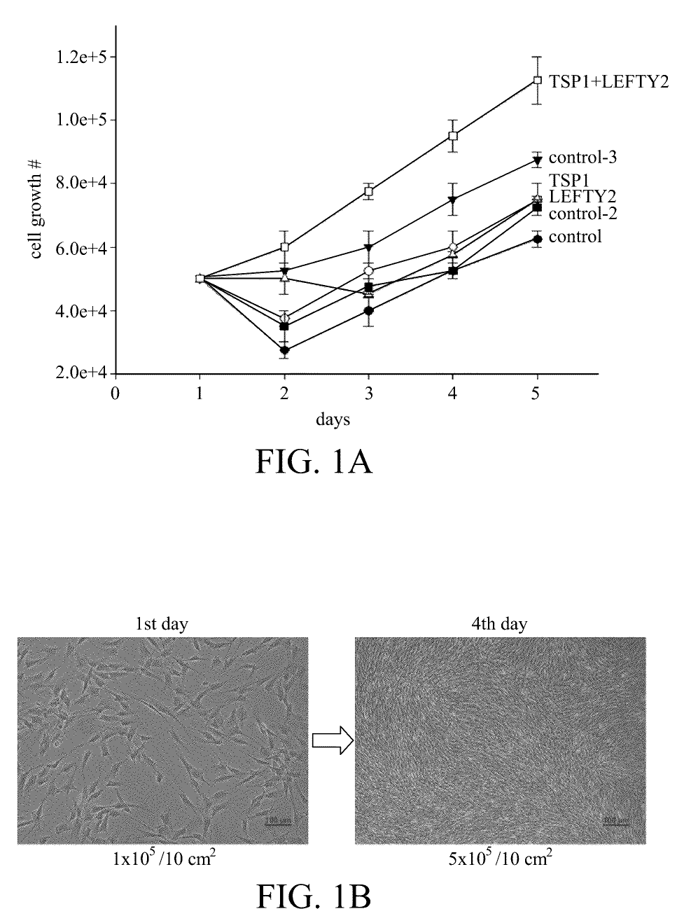

As shown in FIG. 1A, the addition of ADSCs in the TSP1+LEFTY2 test group can promote the growth of stem cells in a relatively short period of time. When cultured at 37° C. and 5% CO₂ until the fifth day, the number of ADSCs added to the TSP1+LEFTY2 test group was about 1.8 times that of the stem cells added to the control group. At the same time, TSP1 and LEFTY2 also showed a synergistic effect on the growth of stem cells. When the stem cells were cultured in the TSP1 test group and the LEFTY2 test group, the performance of stem cell growth was not significantly improved compared with the cells cultured in the control group-2. However, when cells were cultured in the TSP1+LEFTY2 test group, it significantly increased the speed and number of cell growth. As shown in FIG. 1B, ADSCs were cultured with TSP1+LEFTY2, and the growth pattern of stem cells remained similar to that of fibroblasts; and in a culture dish with 1×10⁵ cells per 10 cm², after 4 days of culture, it can be clearly observed that the mesenchymal stem cells grow to 5×10⁵ cells per 10 cm². This result confirms that TSP1 and LEFTY2 have a synergistic effect on promoting stem cell growth, and have a better efficacy improvement than when added alone.

### Example 2: The Medium Supplemented With Medium Additives TSP1 and LEFTY2 Contributes to the Expression of Pluripotency Factors Nanog and OCT-4

As stem cells grow, they tend to differentiate due to their fast-growing nature, reducing the expression of pluripotency factors and eventually differentiating into specific cells. In order to test the influence of TSP1 and LEFTY2 in promoting or maintaining the expression of pluripotency factors in stem cells during the growth of stem cells, ADSCs were cultured at 37° C. and 5% CO² with the control and test media mentioned above and the expression of pluripotency factors in the stem cells was measured.

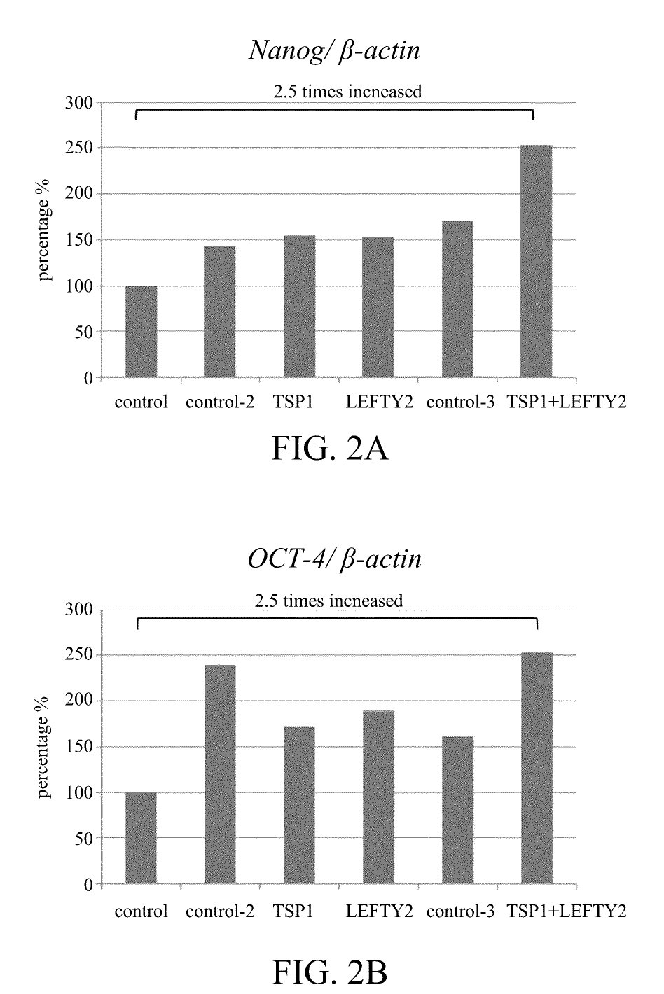

As shown in FIG. 2A, ADSCs cultured with the TSP1+LEFTY2 test set showed a significant increase in the expression of the pluripotency factor Nanog, which increased approximately 2.5-fold compared to the stem cells cultured with the control set. Similar results are shown in FIG. 2B, where the TSP1+LEFTY2 test group significantly increased the expression of pluripotency factor OCT-4 in ADSCs and increased the expression by about 2.5-fold compared to the control cultured stem cells. The results of this experiment confirm that the co-addition of TSP1 and LEFTY2 to stem cells can effectively promote or maintain the expression of pluripotency factors in stem cells, so that the stem cells can grow rapidly while maintaining their stem cell characteristics and not tending to differentiation.

### Example 3: TSP1 and LEFTY2 Medium Additives Did Not Affect the Expression of Antigens on the Surface of Mesenchymal Stem Cells

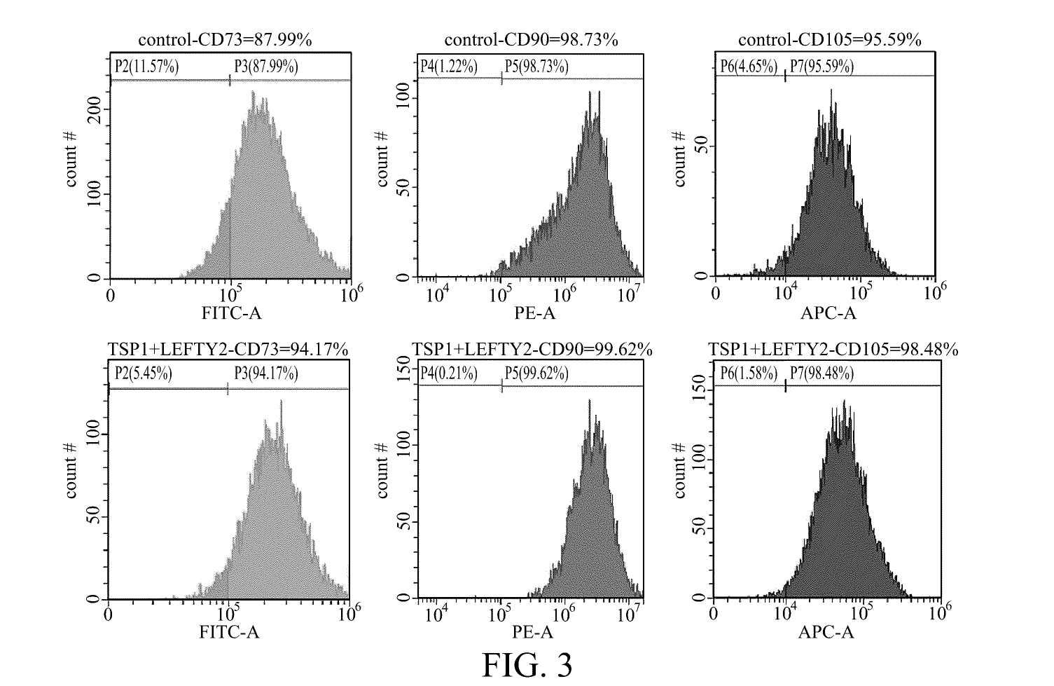

There are various antigens on the surface of adipose-derived mesenchymal stem cells, including CD73, CD90 and CD105. The ADSCs were cultured at 37° C. and 5% CO² with the medium of the control group above and TSP1+LEFTY2 test group to detect whether the expression of various surface antigens in the stem cells was affected. The results are summarized in FIG. 3.

As shown in FIG. 3, CD73, CD90, and CD105 in the ADSCs cultured in the TSP1+LEFTY2 test group had expressions of 94.17%, 99.62%, and 98.48%, respectively. Compared with the control group, the ADSCs cultured in the TSP1+LEFTY2 test group had an increase in the expression of surface antigens by about 5%. The results of this experiment showed that the addition of TSP1 and LEFTY2 did not affect the expression of surface antigens CD73, CD90 and CD105 in stem cells, but also slightly increased the expression of these cell surface antigens.

### Example 4: Mesenchymal Stem Cells Co-Cultured With TSP1 and LEFTY2 Can Effectively Differentiate Into Adipocytes, Osteocytes and Chondrocytes

In order to test whether the addition of TSP1 and LEFTY2 will affect the differentiation ability of stem cells, the ADSCs were cultured with the TSP1+LEFTY2 test group mentioned above, and the subsequent differentiation of the ADSCs was observed.

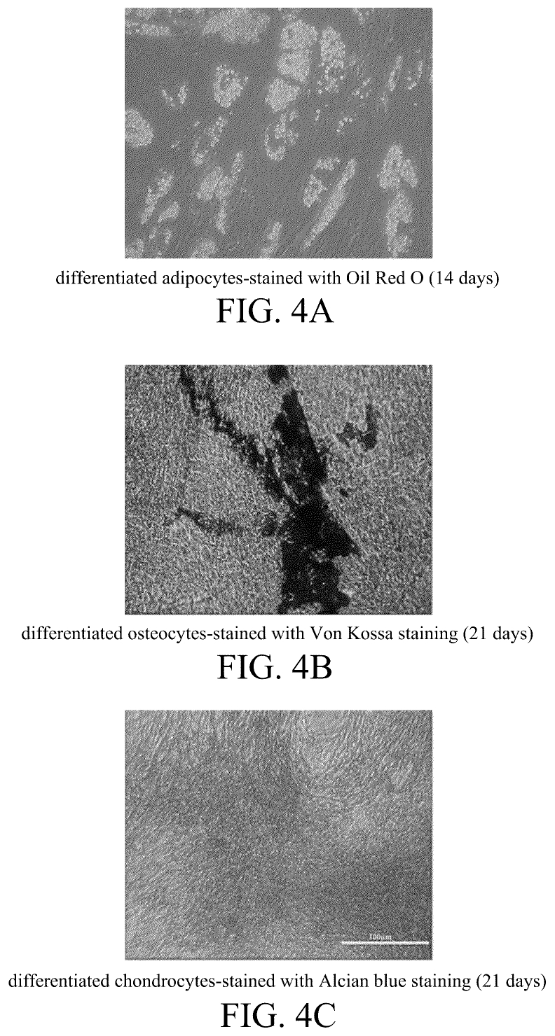

After the TSP1+LEFTY2 test group was cultured at 37° C., 5% CO², for 2 days, the ADSCs differentiated for 14 to 21 days and the types of differentiated cells were observed. As shown in FIG. 4A, 14 days after the differentiation of the ADSCs, and differentiated adipocytes were stained with Oil Red O staining method, and differentiated adipocytes with complete morphology could be found. Similarly, the osteocytes (FIG. 4B) and chondrocytes (FIG. 4C) of the ADSCs mentioned above differentiated for 21 days were stained with Von Kossa staining and Alcian blue staining respectively, and complete differentiated osteocyte and chondrocytes can also be observed. These experimental results show that adding TSP1 and LEFTY2 to stem cells does not affect the relative differentiation ability of the stem cells.

### Example 5: A large Amount of Exosomes Can Be Detected in Stem Cells Cultured by Adding TSP1 and LEFTY2

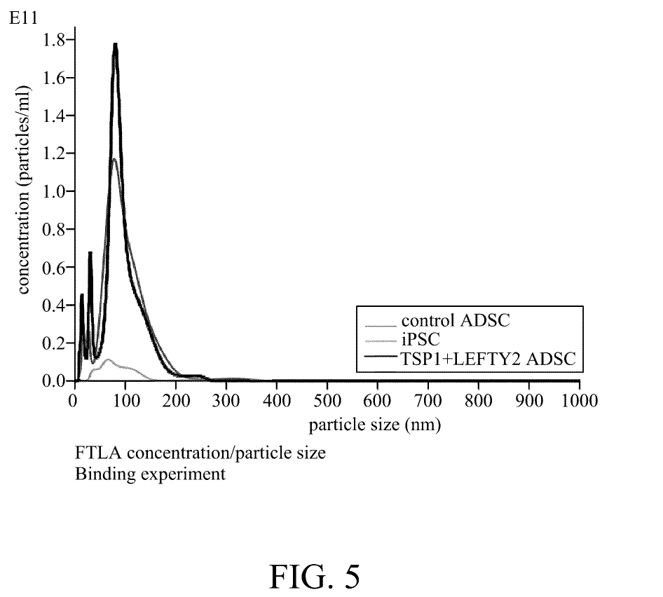

As shown in FIG. 5, the culture medium of the ADSCs cultured by TSP1 and LEFTY2 was collected and exosomes were extracted. The concentration of exosomes secreted by the ADSCs cultured by adding TSP1 and LEFTY2 was 1.04×10¹³±7.34×10¹¹ particles/ml (about 1 billion/c.c), and the control group was 6.46×10¹²±1.30×10¹² particles/ml (about 600 million particles/c.c), while the induced pluripotent stem cell (iPSC) group was 8.28×10¹¹±2.73×10 particles/ml (about 80 million particles/c.c); which is that the amount of exosomes secreted by ADSCs cultured with TSP1 and LEFTY2 was nearly 400 million more than that of the control group, and the amount of exosomes was higher than that of the induced pluripotent stem cell (iPSC) group out nearly 10 times.

### Example 6: The Addition of Medium Additives Such As TSP1 and LEFTY2 Can Effectively Maintain the Number of Neuronal Cells and Effectively Promote the Growth of Neurofilaments

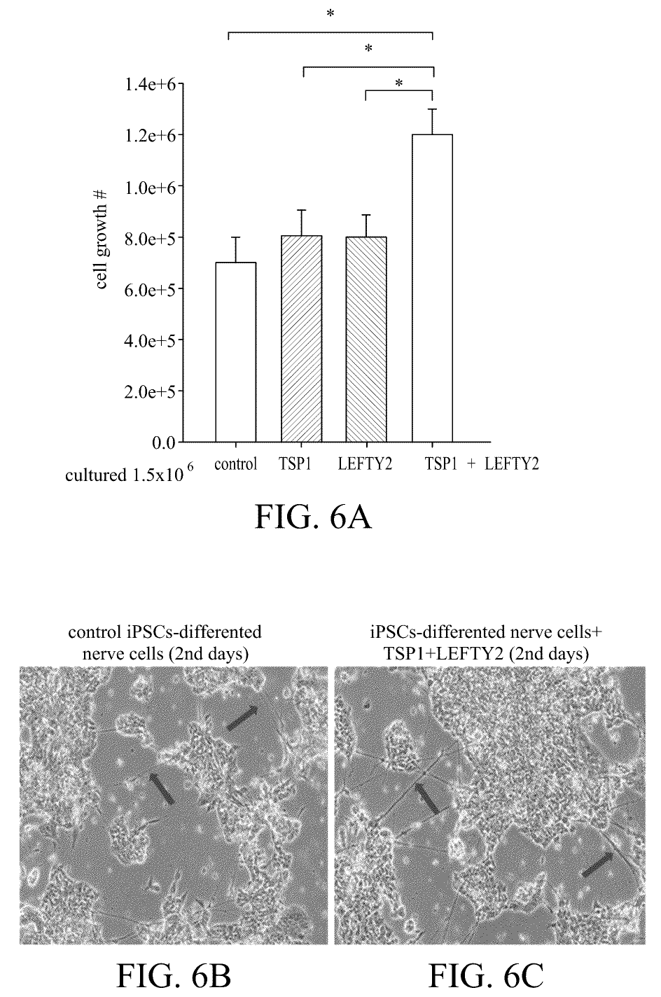

For stem cells, especially induced pluripotent stem cells, the differentiated nerve cells are very easy to die during subsequent culture. The control group mentioned above and different concentrations of the TSP1 test group, LEFTY2 test group, and TSP1+LEFTY2 test group were used to culture the nerve cells differentiated from induced pluripotent stem cells at 37° C. and 5% CO² for 2 days, and test the survival number of cells in each group, the results are organized in FIG. 6.

As shown in FIG. 6A, the differentiated nerve cells cultured in the TSP1 test group and the LEFTY2 test group have slightly improved cell survival rates compared to the control group. After increasing the concentration of the TSP1 test group and LEFTY2 test group, the survival number of differentiated nerve cells further increased, but there was no significant difference compared with the control group (not shown). However, the differentiated neural cells cultured in the TSP1+LEFTY2 test group had a significantly better cell survival rate and compared with the control group. The TSP1 test group and the LEFTY2 test group had a significantly increased number of survival cells. This result shows that TSP1 and LEFTY2 can significantly reduce the death rate of differentiated neuronal cells, and shows that TSP1 and LEFTY2 also have synergistic effects in differentiated stem cells. FIGS. 6B and 6C show the patterns of differentiated nerve cells in the control group and the TSP1+LEFTY2 test group in Example 5. As shown in FIG. 6B, the nerve cells cultured in the control group have shorter neurofilament lengths (arrows) and thinner cell types; while FIG. 6C shows that the nerve cells cultured in the TSP1+LEFTY2 test group have a complete cell type, with long and complete neurofilaments (arrows).

### Example 7: Adding Medium Supplements Such As TSP1 and LEFTY2 Effectively Reduces the Inflammation of Damaged Nerve Cells

In order to further verify the effect of TSP1 and LEFTY2 on reducing the death rate of nerve cells, damaged nerve cells obtained from patients with degenerative diseases were cultured with the culture medium of the control group mentioned above and each test group, and then measured the expression of inflammation-related factors.

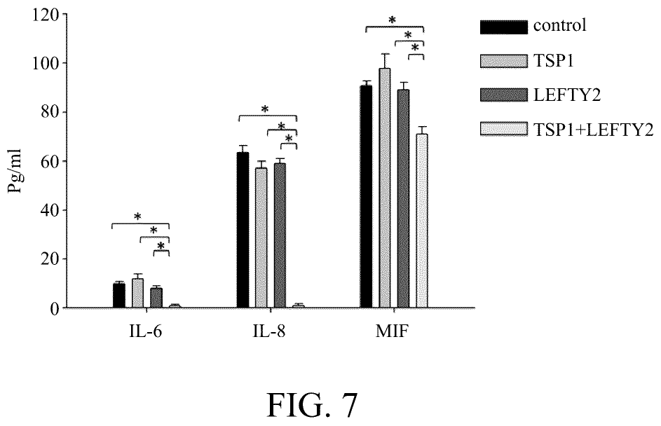

As shown in FIG. 7, the damaged nerve cells cultured with the TSP1+LEFTY2 test group have a significantly reduced expression of inflammatory factors. No matter that it is compared to the control group, the TSP1 test group and the LEFTY2 test group, there is a significant reduction the inflammatory response. In addition, the nerve cells cultured in the TSP1 test group and LEFTY2 test group did not form a significant difference from the control group. This result further confirms that TSP1 and LEFTY2 have a synergistic effect on each other, and have a synergistic effect on the growth of stem cells and the reduction of nerve cell inflammation.

The embodiments mentioned above are only illustrative technical principles, features and effects of the present disclosure, and are not intended to limit the scope of the present disclosure. Anyone skilled in the art can modify and change the embodiments mentioned above without departing from the spirit and scope of the present invention. However, any equivalent modifications and changes accomplished by using the teachings of the present disclosure should still be covered by the scope of the following patent applications. The scope of protection of the present disclosure should be listed in the claims as follows.

What is claimed is:

1. A method for promoting proliferation and expression of pluripotency factors of stem cells, comprising culturing the stem cells in a cell culture medium containing thrombospondin 1 (TSP1) and left-right determination factor 2 (LEFTY2).

2. The method of claim 1, wherein the cell culture medium contains acetylcysteine and vitamin C.

3. The method of claim 1, wherein the stem cells are selected at least one from the group consisting of induced pluripotent stem cells, mesenchymal stem cells, fat stem cells, bone marrow stem cells, peripheral blood stem cells, umbilical cord stem cells and amniotic fluid stem cells.

4. The method of claim 1, wherein the pluripotency factor is selected at least one from the group consisting of Oct4, Sox2, Klf4, c-Myc, Lin28 and Nanog.

5. A method for promoting proliferation of cells differentiated from stem cells, comprising culturing the stem cells in a cell culture medium containing thrombospondin 1 (TSP1) and left-right determination factor 2 (LEFTY2).

6. The method of claim 5, wherein the cells differentiated from the stem cells are nerve cells.

7. The method of claim 5, wherein the cell culture medium reduces an expression of inflammation-related factors in the nerve cells or increases an expression of anti-inflammatory factors in the nerve cells.

8. The method of claim 7, wherein the inflammation-related factor is selected at least one from the group consisting of interleukin-6 (IL-6), interleukin-8 (IL-8), and macrophage migration inhibitory factor (MIF).

9. A composition comprising a stem cell derivative and a pharmaceutically or cosmetically acceptable carrier, wherein the derivative is produced by a method comprising the following steps:
providing a cell culture medium comprising thrombospondin 1 (TSP1) and left-right determination factor 2 (LEFTY2);
culturing the stem cells in the cell culture medium; and
obtaining the derivative from the cell culture medium.

10. The composition of claim 9, wherein the derivative is selected at least one from the group consisting of secretory protein bodies, conditioned medium, exosomes and extracellular vesicles, and the stem cells are selected at least one from the group consisting of induced pluripotent stem cell (iPSCs), mesenchymal stem cells (MSCs, adipose tissue-derived stem cells (ADSCs), bone marrow-derived stem cells (BMSCs, peripheral blood-derived stem cells (PBSCs), umbilical cord-derived stem cells (UCSCs, and amniotic fluid-derived stem cells (AFDSCs).
